(19) United States
(12) Patent Application Publication
(10) Pub. No.: US 2018/0206523 A1
(43) Pub. Date: Jul. 26, 2018

(54) A METHOD OF PROVIDING FUNCTIONAL PROTEINS FROM A PLANT MATERIAL

(71) Applicants: Biotest APS, Middelfart (DK); Aalborg Universitet, Aalborg (DK)

(72) Inventors: Pauli Kiel, Middelfart (DK); Margrethe Anderson, Middelfart (DK); Mette Lubeck, Lyngby (DK)

(73) Assignees: Biotest APS, Middelfart (DK); Aalborg Universitet, Aalborg (DK)

(21) Appl. No.: 15/321,657

(22) PCT Filed: Jun. 25, 2015
(86) PCT No.: PCT/DK2015/050185

§ 371 Date: Dec. 22, 2016

(30) Foreign Application Priority Data
Jun. 26, 2014 (DK) PA201470390

(51) Int. Cl. A23J 1/00; A23J 3/14; B01D 21/26; A23K 10/30; A23K 20/147; A23L 33/185; A23L 33/105; C07K 1/14

(52) U.S. Cl. CPC A23J 1/007; A23J 3/14; B01D 21/262; A23K 10/30; A23V 2002/00; A23L 33/185; A23L 33/105; C07K 1/14; A23K 20/147

(57) ABSTRACT

The present invention relates to the provision of leaf protein concentrate and food grade soluble functional proteins and possibly other high value products and fibres from a green plant material. The method further provides for the possibility obtaining fermentation products as biogas and finally fertilizer. The soluble functional protein product may be used in food and/or in pharmaceutical products.

### TECHNICAL FIELD OF THE INVENTION

The present invention pertains in general to the isolation of leaf proteins for food and feed for monogastric animals preferably organic monogastric animals and particularly soluble functional proteins from a plant material.

### BACKGROUND OF THE INVENTION

The production of animal meat for human consumption plays an important role in the global warming. Especially cattle and other ruminants consume about 6 times more plant protein than they produce animal protein. In additionally the release of methane from the fermentation process in ruminants this factors adds to the “greenhouse effect”. Accordingly, there are very strong arguments for the reduction of ruminant production worldwide—instead the global strategy is to use farm land for plant production in order to simultaneously provide energy and protein for monogastric animals and human consumption. The supply of organic protein feed for monogastric animals (i.e. poultry and pigs) with the right amino acid profile and a competitive price is one of the major challenges for organic agriculture. Local production of suitable feed protein is preferred as substitute for the imported soybean protein. Using nitrogen fixing green plans with high protein content like clover and alfalfa for protein production is in many ways a sustainable solution. By using a sustainable technology without the use of inorganic acids or organic solvents it is possible to obtain an organic protein feed for monogastric animals.

It is well known that plants contain valuable proteins—an example of such a plant is alfalfa (Medicago sativa) and red clover (Trifolium pratense). Like other legumes, its root nodules contain bacteria, Sinorhizobium meliloti, with the ability to fix nitrogen. Accordingly the leaves contain high levels of protein regardless of the amount of available nitrogen in the soil. The nitrogen-fixing abilities of alfalfa improve agricultural efficiency as it provides a high yield of protein per hectare and additionally increases soil nitrogen. Alfalfa is primarily used as feed for dairy cattle due to its protein content and level of digestible fibres. In addition alfalfa sprouts are used for human consumption for example in salads and sandwiches. Dehydrated alfalfa leaf is commercially available as a dietary supplement in several forms, such as tablets, powders and tea.

Extract of alfalfa is generally recognized as safe (GRAS) by FDA (GRAS—182.20). Alfalfa is preferred for the production of biofuels, compared to maize, potatoes, sugar beet and winter wheat due to its low consumption of diesel, fertiliser and pesticides. Moreover it provides a satisfying energy output.

Along the increased need for vegetable proteins another need has emerged simultaneously—the need for environmental friendly combustion fuels. Limited recourses of fossil fuels and an increasing demand for energy in the industrial countries as well as in a growing number of fast developing countries has resulted in an increased focus on alternatives to fossil fuels as energy sources. In addition, fossil fuels contribute significantly to the amount of CO2 released into the atmosphere and therefore also contribute significantly to the global warming. Consequently, the demand for alternative- and environmental friendly combustion fuels is evident.

In order to simultaneously meet the increasing need for organic protein feed and vegetable proteins for food applications and the environmental friendly combustion fuels the industry has developed alcohol fermentation processes where proteins contained in the remaining fermentation effluent are recovered after the fermentation process and subsequently utilised as an animal feed. These proteins are however low value proteins.

For example Gibbons et al. (1987) discloses a semi-continuous diffusion fermentation process for obtaining ethanol and cubed feed protein from fodder beets. The process is performed in a special shaped fermentor wherein ethanol is continuously exiting from one end of the fermentor and the fermentation effluent is continuously recovered at the other end in form of a cubed protein feed. Accordingly there is a need for a more effective process, which simultaneously provides functional proteins and possible other high value products fermentation products from the plant material.

The traditional alcohol fermentation processes endure from inefficiencies in utilizing the plant material in an optimal energy effective way, disclosing the need for an additional and more effective process, which may simultaneously provide functional proteins product or high value feed protein for monogastric animals, combined with high value products and fermentation products from a plant material. Accordingly, the process of the invention will not only provide improved process economy, but also a more sustainable utilization of agricultural plant materials.

### SUMMARY OF THE INVENTION

In a first aspect of the present invention relates to a method for providing a chlorophyll concentrate and at least one soluble functional protein said method comprises the steps of:
(i) pressing a green plant material,
(ii) obtaining a press cake and a green juice
(iii) subjecting said green juice to UHT Sterilization/pasteurization and obtaining a sterilized green juice,
(iv) subjecting the sterilized/pasteurized green juice to separation and obtaining a chlorophyll concentrate and a clear juice,
(v) recovering at least one soluble functional protein from the clear juice.

Please also see FIG. 1.

In yet an aspect the present invention pertains to a method for providing a leaf protein concentrate said method comprises the steps of:
(i) pressing a green plant material,
(ii) obtaining a press cake and a green juice
(iii) optionally subjecting said green juice to UHT Sterilization/pasteurization and obtaining a sterilized/pasteurized green juice
(iv) subjecting the green juice or the sterilized/pasteurized green juice to lactic acid fermentation followed by sedimentation and separation,
(v) obtaining a leaf protein concentrate and a brown juice.

Please also see FIG. 2.

In another aspect the present invention (FIG. 1) pertains to a method for providing at least one soluble functional protein said method comprises the steps of (i) disintegrating and pressing a plant material, (ii) obtaining a press cake (I) and a green juice (II), (iii) subjecting said green juice to UHT Sterilization/pasteurization and obtaining a sterilized/pasteurized green juice, (iv) subjecting the sterilized/pasteurized green juice to separation (such as but not limited to centrifugation and/or microfiltration) obtaining a chlorophyll concentrate and a clear juice (v) recovering at least on soluble functional protein and a clear juice.

In another aspect the present invention (FIG. 2) pertains to a method for providing a leaf protein feed concentrate said method comprises the steps of (i) disintegrating and pressing a plant material, (ii) obtaining a press cake (I) and a green juice (II), (iii) optionally subjecting said green juice to UHT Sterilization/pasteurization and obtaining a sterilized/pasteurized green juice, (iv) subjecting the green juice or the sterilized/pasteurized green juice to lactic acid fermentation followed by sedimentation and separation, (v) obtaining a leaf protein feed concentrate and a brown juice.

In a further aspect the present invention (FIG. 1) pertains to the at least one soluble functional protein obtainable by the method of the present invention and to the use of such protein as a feed ingredient, a pharmaceutical ingredient, a cosmetic ingredient or as a vegetable protein.

In yet a further aspect the present invention (FIG. 2) pertains to the green leaf protein concentrate and the brown juice obtainable by the method of the present invention and to the use of these phases as animal feed or for further isolation of high value products and biogas production.

In an aspect the present invention pertains to a fermentation product obtainable by the method of the present invention. Also the present invention pertains to a fermentation effluent and the use of such effluent in biogas fermentation.

In another aspect the present invention pertains to the use of the effluent from the biogas production as a fertilizer and the use of such in crop production.

### BRIEF DESCRIPTION OF THE FIGURES

The invention may be described in the following non-limiting figure.

FIG. 1 shows a method for providing a press cake, a chlorophyll concentrate, at least one soluble functional protein, a fermentation product comprising e.g. amino acids, organic acids, methane and/or ethanol and a fermentation effluent which may be used as a fertilizer.

FIG. 2 shows a method, especially suitable for organic feed protein production e.g. for monogastric animals. The method provides a press cake, a green, leaf protein concentrate, a fermentation product such as biogas and a fermentation effluent which may be used as a fertilizer.

FIG. 3 show the nitrogen solubility of acid precipitated alfalfa soluble functional protein.

### DETAILED DESCRIPTION OF THE INVENTION

It is the aim of the present invention to provide a green biorefinery including a method for isolating functional proteins and possibly other high valuable products before subjecting the residue (e.g. the brown juice) to a fermentation process.

The inventors found that there is a large potential for recovering functional proteins with valuable properties from plants (such as alfalfa). Furthermore, the inventors found that the functional proteins with advantage can be isolated from the liquid (i.e. the clear juice) of the processed plant materials while other higher value products can be obtained from the press cake and the chlorophyll concentrate. The remaining liquid (i.e. the brown juice) can subsequently be used as a fermentation medium in a fermentation process.

Thus, the invention is based on a combination of three main process steps, namely a first step where the plant material (preferably freshly harvested) is disintegrated by fine cutting followed by pressing and separation in a green juice and a press cake (I) as this facilitates the present process. In a preferred embodiment the green juice is UHT pasteurized/sterilized, which reduces the number og microorganisms and surprisingly also make it easier to separate the green juice e.g. by centrifugation into a, chlorophyll concentrate (II) and a clear juice. The soluble functional proteins may be recovered from the clear juice resulting in a brown juice having a high content if fermentable compounds and a low content of proteins and nitrogen. Following protein recovery, the brown juice can be used for isolation of phytochemicals and/or used as fermentation medium in a fermentation process.

Accordingly, a first aspect of the present invention relates to a method for providing a chlorophyll concentrate or at least one soluble functional protein said method comprises the steps of:
(i) pressing a green plant material,
(ii) obtaining a press cake and a green juice
(iii) subjecting said green juice to UHT Sterilization/pasteurization and obtaining a sterilized green juice,
(iv) subjecting the sterilized/pasteurized green juice to separation and obtaining a chlorophyll concentrate and a clear juice,
(v) recovering at least one soluble functional protein from the clear juice

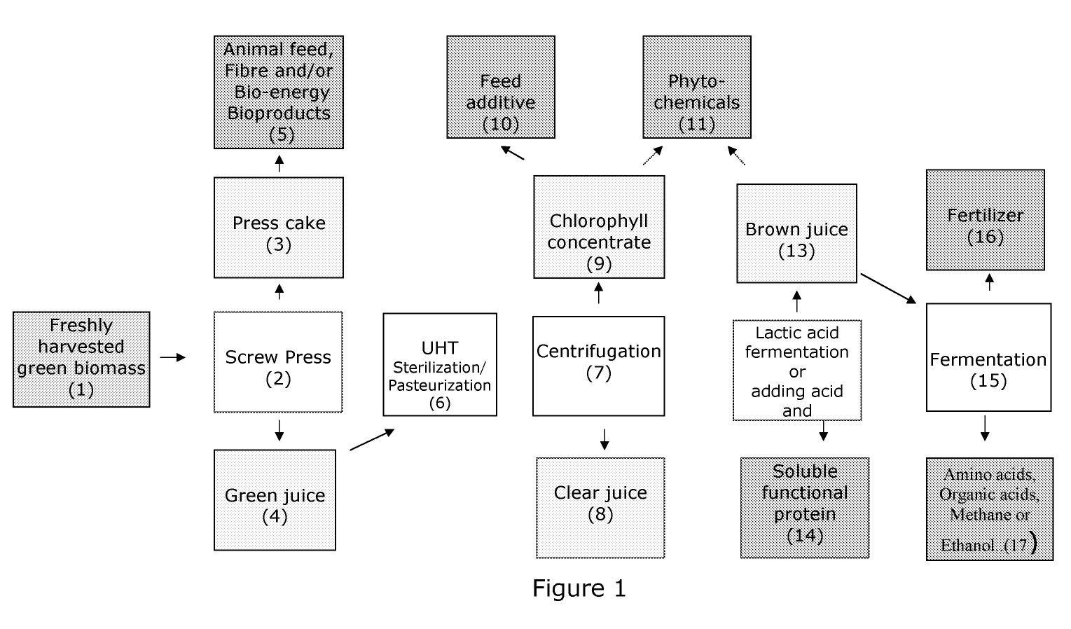

Another aspect of the present invention (FIG. 1) pertains to a method for providing at least one soluble functional protein, the method comprises the steps of (i) disintegrating and pressing a plant material, (ii) obtaining a press cake (I) and a green juice (iii) subjecting said green juice to UHT sterilization/pasteurization and obtaining a sterilized/pasteurized green juice (iv) subjecting said sterilized/pasteurized green juice to separation (such as but not limited to centrifugation and/or microfiltration) and obtaining a chlorophyll concentrate and a clear juice and (v) recovering at least one functional protein from the clear juice.

In yet another aspect the present invention pertains to a method for providing at least one soluble functional protein, the method comprises the steps of (i) disintegrating and pressing a plant material, (ii) obtaining a press cake (I) and a green juice (iii) subjecting said green juice to UHT sterilization/pasteurization and obtaining a sterilized/pasteurized green juice (iv) subjecting said sterilized/pasteurized green juice to separation (such as but not limited to centrifugation and/or microfiltration) and obtaining a chlorophyll concentrate (II) and a clear juice, (v) recovering at least one functional protein from the clear juice and wherein (v) is preferably conducted at a temperature at or below 10° C.

In a further aspect the present invention pertains to a method for providing a (organic) leaf protein feed concentrate said method comprises the steps of:
(i) pressing a green plant material,
(ii) obtaining a press cake and a green juice
(iii) optionally subjecting said green juice to UHT Sterilization/pasteurization and obtaining a sterilized/pasteurized green juice
(iv) subject the green juice or the sterilized green juice to lactic acid fermentation followed by separation,
(v) obtaining a leaf protein feed concentrate and a brown juice.

In an embodiment step (i)-(ii) is conducted at a temperature at or below 30° C., such as at or below 29° C., e.g. at or below 25° C., such as at or below 20° C., e.g. at or below 15° C., such as in the range from 1-15° C., e.g. in the range from 16-20° C., such as in the range from 21-25° C., e.g in the range from 25-30° C., preferably in the range from 15-29° C.

A brown juice is obtained following protein recovery. This juice may be applied in a fermentation process, thus proving at least one fermentation product. In a further embodiment the method of the present invention comprises a step of fermentation of the brown juice and obtaining a fermentation product and a fermentation effluent.

The press cake (I) can be ensiled or dried and used as animal feed. Alternatively the press cake (I) can be used for the production of fibres. The press cake (I) and/or the residues from this phase following fibre production can be used in a 2nd generation biorefinery for the production of different fermentation products like enzymes, bio-fuel, biogas and fertilizers.

The chlorophyll concentrate (II) can be used for further isolation and recovery of high valuable products or be ensiled or dried and used as animal feed additive. Alternatively the chlorophyll concentrate (II) and/or the residues from these phases following recovery of high valuable products can be used in a 2^{nd} generation biorefinery for the production of different fermentation products like enzymes, bio-fuel, biogas and fertilizers.

The unique combination of using freshly harvested, fine cut green plant materials and lactic acid fermentation of the green juice followed by separation at a temperature preferably below 30° C. provides (i) a high yield of preserved, high value feed protein for monogastric animals containing living lactic acid bacteria with pro-biotic activity i the animals, a press cake useful as cattle feed and a brown juice useful as fermentation medium, followed by recirculation of the inorganic compounds to the fields as fertilizer.

Plant Material

All kinds of fresh plant materials from various plant species and genera may be useful in the method of the present invention.

However it is presently preferred to employ plant materials that are generally considered as safe for humans (GRAS).

As one of the objects of the present invention is to provide plant material derived proteins, it is preferred to use plant materials with a high content of proteins. Hence, in a useful embodiment of the present invention the plant material has a protein content of at least 0.1% (w/w), e.g. least 0.2% (w/w), such as 0.5% (w/w), including least 1% (w/w), such as at least 2% (w/w), e.g. at least 3% (w/w), including at least 4% (w/w), e.g. at least 5% (w/w), such as at least 6% (w/w).

In further embodiments, the plant material has a protein content in the range of 0.1%-6% (w/w), such as in the range of 0.2%-5% (w/w), e.g. in the range of 0.3%-4% (w/w), such as in the range of 0.4%-3% (w/w), e.g. in the range of 0.5%-2% (w/w), such as in the range of 1.5%-4% (w/w), including in the range of 1%-3% (w/w), e.g. in the range of 2%-5% (w/w), such as in the range of 2%-3% (w/w), e.g. in the range of 0.1%-2.5% (w/w), including in the range of 1%-2% (w/w).

Even though the method of the present invention is applicable to any kind of plant material it may be useful to employ perennial plants with high biomass yield and/or low cost plant materials in order to reach an improved process economy.

In an embodiment of the present invention the plant material is selected from alfalfa, clover, grass, beet, chicory, Jerusalem artichoke, sugar cane, carrot, radish, roadside crops and combinations hereof.

In a preferred embodiment the plant material is alfalfa (Medicago sativa).

The green part of the plants are preferred however all parts of the plants may be used. Thus, in an embodiment the plant material comprises a plant part selected from the group consisting of stem, leaves, root, fruits, tubers and combinations thereof.

Harvest, Cutting, Transport and Storage of Green Plant Material

In accordance with the present invention, the green plant material is harvested e.g. by cutting before being transported to the pressing plant. In the time from harvest and until transport to the pressing plant the green plant material may be left in swaths followed by transport to the pressing plant preferably as fast as possible.

It is preferred that the green plant material after harvest and transport to the pressing plant is stored in a cold place for as short as possible time and not more than few hours after harvest and transportation and before pressing.

The plant material is fine cut preferable immediately before pressing.

Thus, in a preferred embodiment the time from harvest of the plant material to pressing of the green plant material is at the most 3 hours, such as 2.5 hours, such as 2 hours, such as 1.5 hours, e.g. 1 hour, such as 30 minutes, e.g. 15 minutes.

In a further preferred embodiment the storage temperature of the green plant material is preferably below 20° C., such as below 18° C., e.g. below 15° C., such as below 10° C., e.g. below 8° C., such as below 6° C., e.g. below 5° C., such as below 4° C., e.g. below 3° C., such as below 2° C., e.g. below 1° C., but higher than 0° C., in order to avoid spoilage. Cutting the plant materials will result in activation of enzymes. In order to prevent the activation of primarily hydrolytic enzymes such as proteolytic enzymes the fine cutting is carried out immediately before pressing. The proteolytic enzymes will split the native proteins in peptides and free amino acids that not can be precipitated at pH 4. In order to prevent enzymatic degradation and microbial spoilage of the material is important to keep the material at a low temperature, but higher than 0° C.

Disintegration of the Plant Material and Separation into a Press Cake (I) and a Green Juice.

In accordance with the present invention, the plant material is subjected to a mechanical process, ex. Vincent Screw press for the purpose of open up the plant cells to make the proteins available for recovery and the carbohydrates available for fermentation. Furthermore, the mechanical process or the disintegration of the plant material results in a composition comprising a green juice and press cake (I).

The plant material can be processed by a variety of well-known processes which results in an efficient opening of the plant cells. Such efficient mechanical processes include grinding, milling, hacking, squeezing, slicing, abrading, pressing, crushing, chipping, refining and combination thereof.

In a preferred embodiment the mechanical process is a Vincnet screw press or a refiner. Preferably the refiner work with a rotation speed in the range from 1000-1500 rpm.

An efficient mechanical process useful in the method according to the invention, is one which efficiently enhance I) the overall surface area to mass ratio in order to enable degradation of the material into a satisfactory level, and II) cell opening thus providing an efficient release of cell juice containing carbohydrates and proteins. In a useful embodiment, the pressing and disintegration process is carried out in a patented Vincent Screw press or a two-step grinding process which results in an even more efficient release of proteins and carbohydrates.

Temperature is of significant to the present invention, as high temperatures lead to denatured proteins (e.g. proteins altered in the native 3-D structure). Proteins isolated after precipitation by heat are denatured and thus only valuable as animal feed. Accordingly, such proteins cannot be used for purposes where the properties of native (i.e. functional) proteins are needed.

Prior to disintegration the plant material may be washed preferably in cold water. The washing serves to remove impurities and to lower the temperature of the plant material. To avoid spoilage it may be contemplated that the plant material has a temperature at or below 10° C., such as below 9° C., e.g. below 8° C., such as below 7° C., e.g. below 6° C., such as below 6° C., e.g. below 5° C., such as below 4° C., e.g. below 3° C., such as below 2° C., e.g. below 1° C. before disintegration

Water remaining on the plant material after washing may be removed by separation—preferably at low speed.

Thus, in an embodiment the plant material is washed and cooled to a temperature at or below 10° C., e.g. below 9° C., such as at or below 8° C., e.g. at or below 7° C., such as at or below 6° C., e.g. at or below 5° C., such as at or below 4° C., e.g. at or below 3° C., such as at or below 2° C., such as in the range from 2−10° C., e.g. in the range from 3−9° C., such as in the range from 4−8° C., e.g in the range from 5−7° C., such as in the range from 6−7° C., preferably in the range from 2−5° C.

All temperatures mentioned in the present application refer to temperatures relative to the local atmospheric or ambient pressure.

In a useful embodiment the disintegration and/or pressing processes are kept between 5 and 30° C., such as between 6-29° C., e.g. between 7-28° C., such as between 8-27° C., e.g. between 9-26° C., such as between 10-25° C., e.g. between 11-24° C., such as between 12-23° C., e.g. between 13-22° C., such as between 14-21° C., e.g. between 15-20° C., such as between 16-19° C., e.g. between 17-18° C.

During the above described processing of the plant material, enzymes may be added in order to obtain an at least partial hydrolysis of pectin, cellulose and other carbohydrates in the plant material resulting in a processed material containing an increased amount of microbially fermentable sugars which are used in the subsequent alcohol fermentation. Besides from increasing the amount of microbially fermentable sugars, the at least partial hydrolysis of pectin may also lead to the liberation of pectin bound protein. In a useful embodiment, the enzyme is added to the processed plant material, e.g. after the processing of the material.

In preferred embodiments, the at least one enzyme added during the disintegration of the plant material and/or to the disintegrated plant material (i.e. the liquid phase and the solid phase) is selected from a group consisting of cellulase, kitinase, β-fructosidase, β-glucanase, hemicellulase, xylanase, invertase, glactosidase, polygalacturonase, xylosidase and arabinosidase. In useful embodiments, two or more enzymes, such as three or more enzymes, four or more enzymes or five enzymes or more enzymes, are added to the plant material during disintegration and/or to the disintegrated plant material. Under some circumstances it may be useful to add the two or more enzymes together or subsequently during the disintegration of the plant material and/or to the disintegrated plant material.

In useful embodiments, the enzyme is added to the plant material and/or disintegrated plant material in a quantity of at least 1 ng per kg material dry weight, such as at least 5 ng per kg material dry weight, e.g. 10 ng per kg material dry weight, including at least 25 ng per kg material dry weight, such as at least 50 ng per kg material dry weight. The amount of the enzyme added to the plant material and/or processed plant material is an amount which results in the presence in the material of 10 to 5000 units per gram material, such as in the range of 100 to 3000 units per gram material, including in the range of 250 to 2500 units per gram material, such as in the range of 500 to 1000 units per gram material, including in the range of 750 to 1000 units per gram material. In the present context, the term “units” relates to the activity of an enzyme and is defined as μmoles of substrate reacted per minute per gram of the measured sample at fixed standard conditions.

During the above described processing of the plant material it may be useful to prevent enzymatic browning. Accordingly, sulphite (K₂S₂O₅) may be added during the disintegration of the plant material and/or to the disintegrated plant material and/or to the green juice. In a preferred embodiment 0.003% w/w sulphite, including 0.004% (w/w) sulphite is added during the processing of the plant material and/or to the processed plant material and/or to the plant juice, such as 0.005% (w/w) sulphite, including 0.006% (w/w) sulphite, such as 0.007% (w/w) sulphite, including 0.008% (w/w) sulphite, such as 0.009% (w/w) sulphite, including 0.010% (w/w) sulphite, such as 0.011% (w/w) sulphite, including 0.012% (w/w) sulphite, such as 0.013% (w/w) sulphite, including 0.014% (w/w) sulphite, such as 0.015% (w/w) sulphite, including 0.016% (w/w). In a useful embodiment, the addition of sulphite is combined with the lowering the pH of the green juice.

In preferred embodiments sulphite in the range of 0.003% (w/w)-0.016% (w/w), such as in the range of 0.005% (w/w)-0.010% (w/w), e.g. in the range of 0.009% (w/w)-0.015% (w/w), such as in the range of 0.004% (w/w)-0.012% (w/w) is added during the disintegration of the plant material and/or to the disintegrated plant material and/or to the green juice.

Following disintegration the plant material is separated into a press cake (I) and a green juice by pressing e.g. in a screw press. Preferably the pressing is conducted by applying a pressure in the range from 40-300 Nm, such as in the range from 50-250 Nm, e.g. in the range from 60-200 Nm, such as in the range from 70-150 Nm, e.g. in the range from 80-100 Nm.

The press cake (I) of the plant material comprises fibres of cellulose, hemicelluloses, pectin and lignin. Green juice comprises small fibres, cell debris and chloroplasts in suspension, whereas part of the proteins as well as organic acids, amino acids, peptides and salts are in solution.

The green juice obtained following pressing preferably has a protein content in the range of 0.1 to 15% (w/w), such as in the range from 0.2 to 14%/w/w), e.g. in the range from 0.3 to 13% (w/w), such as in the range from 0.4 to 12%/w/w), e.g. in the range from 0.5 to 11% (w/w), such as in the range from 1 to 10%/w/w), e.g. in the range from 2 to 9% (w/w), such as in the range 3 to 8%/w/w), e.g. in the range from 4 to 7% (w/w), such as in the range from 5 to 5%/w/w).

Preferably the green juice is cooled to between 2-5° C., such as between 2.5-4.5° C., e.g. between 3-4° C., such as between 3.5-5° C. in order to prevent spoiling of proteins and other organic compounds in the juice.

Due to its contents the press cake (I) and the chlorophyll concentrate may be subjected to one or more isolation process thus obtaining at least one high value product. The high value product may be selected from the group consisting of fibres, such as cellulose, hemicelluloses and ligning, proteins, pigments such as chlorophyll, xanthophylls, β-carotene, anthocyanins, cryptoxanthin, violaxanthin, zea xanthin and neoxanthin, phytosterols such as α- and β-sitosterol, campesterol, α- and β-spinasterol, stigmasterol, cycloartenol and esters thereof, coumarins such as medicagol, coumestrol, savitol, trifoliol, lucernol, triterpene saponins such as medicagosides, medicagenic acid, soyasapogenols, hederagenin, flavonoids such as isoflavonoids, isoflavons such as tricin, biocanin, diadzein, formononetin, genistein, coumesterol, sativan, 5′methoxy-sativan, antibiotics, antiinflamatory compounds and immune system enhancing phytoestrogens.

In a preferred embodiment the at least one high value product is at least one soluble functional protein.

The press cake (I) may also be used as an animal feed (e.g. fodder pellets or silage) or in a fermentation process (please see below).

Separation of the Green Juice into a Green, Chlorophyll Concentrate (II) and a Clear Juice

The suspended materials in the green juice can be separated from the clear solution of proteins and low molecular weight molecules by separation. In the present context the separation may be selected from the group consisting of centrifugation, microfiltration or combinations thereof.

After UHT sterilization/pasteurization the juice is separated by centrifugation, microfiltration or combinations thereof.

Under such separation steps the temperature is preferably between 2-5° C., such as between 2.5-4.5° C., e.g. between 3-4° C., such as between 3.5-5° C. in order to prevent spoiling of proteins and other organic compounds in the juice.

Preferably the separation is performed using centrifugation. The centrifugation may be followed by microfiltration (please see next part regarding separation of the green supernatant into a solid phase (III) and a clear juice).

The centrifugation is advantageously performed at 2-10.000×g, such at 3-9.000×g, e.g. 4-8.000×g, such at 5-7.000×g, e.g. 6-7.000×g.

The green supernatant preferably has protein content in the range of 0.1 to 15% (w/w), such as in the range from 0.2 to 14%/w/w), e.g. in the range from 0.3 to 13% (w/w), such as in the range from 0.4 to 12%/w/w), e.g. in the range from 0.5 to 11% (w/w), such as in the range from 1 to 10%/w/w), e.g. in the range from 2 to 9% (w/w), such as in the range 3 to 8%/w/w), e.g. in the range from 4 to 7% (w/w), such as in the range from 5 to 5%/w/w).

Freezing and Thawing

In alternative to UHT sterilization/pasteurisations in step (iii) is to freeze and thaw the green juice prior to step (iv). The green juice may thawed and preserved at a temperature in the range from 2 to 5° C., such as in the range from 2.5 to 4.5° C., e.g. in the range from 3 to 4° C. until centrifugation. Centrifugation of the thawed green juice provides a clear juice similar to the clear juice obtained above.

Accordingly, the method comprises the steps of (i) disintegrating and pressing a plant material, (ii) obtaining a first solid phase (I) (i.e. the press cake) and a green juice (iii) freezing and thawing said green juice to a temperature in the range from 2 to 5° C., (iv) subjecting said thawed green juice to separation and obtaining a chlorophyll concentrate and a clear juice, (v) recovering at least one soluble functional protein from the clear juice.

In an embodiment step (ii)-(v) is conducted at a temperature at or below 10° C.

UHT Sterilization/Pasteurization of the Green Juice

In order to avoid spoilage of the green juice, the green leaf protein, the clear juice and finally the isolated functional protein as well as contamination of a (batch or continuous) lactic acid fermentation process the green juice is UHT sterilized/pasteurized immediately after production.

In an embodiment the lactic acid fermentation comprises adding Lactobaciluus salivarius (e.g. strain BS 1001) to the green juice following UHT sterilization/pasteurization. In a preferred embodiment any probiotic lactic acid bacterial may be added, such as Lactobacillus paracasei, Lactobacillus plantarum or Lactobacillus delbrueckii. In bach culture an addition of an overnight pre-culture of 5% will be sufficient using fresh green juice. In continuous culture the pasteurization is needed to prevent other bacteria in taking over and contamination of the protein product.

The inventors surprisingly found that the UHT sterilization/pasteurization in addition to the preservation makes it easier to remove the suspended material (chlorophyll concentrate) from the dissolved material (clear juice) by separation.

The UHT sterilization/pasteurization is preferably carried out at temperatures from 72° C. to 150° C. in 0.07 to 15 seconds achieving products with low to zero content of living microorganisms and low changes of the structure and functionally of the proteins and other chemical compounds in the material.

Accordingly, it is appreciated that the temperature of green juice, the green supernatant and/or the clear juice is between 2-5° C., such as between 2.5-4.5° C., e.g. between 3-4° C., such as between 3.5-5° C. in order to prevent spoiling of proteins and other organic compounds in the juice.

In a particular preferred embodiment the green supernatant is obtained using separation, the clear juice is obtained using microfiltration and the temperature of the green juice (before separation), the green supernatant and the clear juice is below 10° C., e.g. below 9° C., such as at or below 8° C., e.g. at or below 7° C., such as at or below 6° C., e.g. at or below 5° C., such as at or below 4° C., e.g. at or below 3° C., such as at or below 2° C., e.g. at or below 1° C., such as in the range from 2-5° C., such as in the range from 2.5-4.5° C., e.g. in the range from 3−4° C., such as in the range from 3.5-5° C.

Such combination provides a particular high yield of functional proteins having preserved a high activity.

Precipitation and Recovery of the Protein

Following the separation the clear juice is subjected to a protein recovery process in order to obtain a high-value protein, preferable a native protein having preserved biological function (i.e. functional proteins) e.g. a soluble protein.

In accordance with the invention, useful “native proteins” or “functional proteins” or “soluble proteins” includes proteins which have preserved at least one of the properties selected from the group consisting of protein activity, protein solubility, gelatinizing, water absorption, oil absorption, emulsifying, and foaming properties.

Proteins isolated after precipitation by heat combined with low pH are partly denatured and are only valuable as animal feed and thus not for purposes where the function is needed. Accordingly, denatured or partly denatured proteins, which are considered as feed grade proteins, are of lower priority in the present invention, but still a product obtainable by the present invention. Denaturation of proteins involves the breaking of many of the weak linkages, or bonds (e.g. hydrogen bonds) within a protein molecule that are responsible for the highly ordered structure of the protein in its natural (native) state. Denatured proteins have a looser, more random structure and most are insoluble and have lost some or all of their functions.

In preferred embodiments, the proteins are recovered by precipitation, ultrafiltration, chromatography or a combination thereof.

Such precipitation includes but is not limited to acid precipitation.

In a preferred embodiment the acid precipitation is performed by the addition of at least one acid, such as an organic acid or inorganic acid or combinations thereof. Examples of useful protein precipitating acids are CH3CHOHCOOH, CH3COOH, HCOOH, H2SO4, HNO3, and H3PO4. In order to obtain a satisfactory precipitation is preferred that the pH of the clear juice is adjusted to a pH in the range from 2 to 5, such as in the range from 2.5 to 4.5, e.g. in the ranger from 3 to 4, e.g. in the range from 3.5 to 4.

The pH is preferably adjusted to provide isoelectric point precipitation. In an embodiment the at least one isolated functional protein is purified by washing with water adjusted to the isoelectric point pH. The at least one isolated functional protein may further be purified by dissolution of the protein at pH 9.0 followed by precipitation once more at the isoelectric point.

Thus depending on the particular isolated functional protein, such protein may be dissolved at certain pH values, such as higher than pH 8.0 and lower than 2.0 and precipitated at the isoelectric point of the protein, such as pH 4.0.

The precipitated protein may be recovered (isolated) from the clear juice by means of separation selected from the group consisting of centrifugation, filtration, decanting and combination hereof.

It is preferred that the function of the protein is preserved during the various process steps. Thus in an embodiment the at least one isolated functional protein has preserved at least 50%, such as at least 60%, e.g. at least 70%, such as at least 75%, e.g. at least 80%, such as at least 85%, e.g. at least 90%, such as at least 95%, e.g. at least 97%, such as at least 98% or e.g. at least 99% of the activity of the natural protein.

The protein content in the green juice, the green supernatant and the clear juice may be measured as N (Kjeldahl nitrogen) times 6.25 often named crude or raw protein. In the present context the terms “protein content”, “raw protein” and “crude protein” are used herein interchangeably.

Likewise the yield of functional protein compared to the initial protein present in the initial plant material may be calculated as stated in e.g. Example 1 and Example 4.

Proteins can be characterized by e.g. X-ray crystallography, Nuclear Magnetic Resonance, Cryo-electron microscopy, Circular dichroism or combinations hereof. If the protein for instance is an enzyme, the activity can be measured as either the consumption of substrate or production of product over time. A large number of different methods of measuring the concentrations of substrates and products exist in the art and many enzymes can be assayed in several different ways such as but not limited to initial rate expression, progress curve experiments, transient kinetics experiments and/or relaxation experiments. Enzyme assays can be split into two groups according to their sampling method: continuous assays (e.g. spectrophotometric, fluorometric, calorimetric and/or chemiluminescent), where the assay gives a continuous reading of activity, and discontinuous assays (e.g. radiometric and/or chromatographic), where samples are taken, the reaction stopped and then the concentration of substrates/products determined.

The at least one isolated functional protein obtainable by the method according to the present invention may be used as a food additive—e.g. to provide water absorption, fat absorption, emulsifying, gelatizing or foaming.

Moreover the at least one isolated functional protein obtainable by the method of the present invention may be used as a vegetable protein.

Use of Chlorophyll Concentrate.

Due to its contents the chlorophyll concentrate may be subjected to one or more isolation processes thus obtaining at least one high value product. The high value product may be selected from the group consisting of fibres such as cellulose, hemicelluloses, ligning, proteins, pigments such as chlorophyll, xanthophylls, β-carotene, anthocyanins, cryptoxanthin, violaxanthin, zea xanthin, neoxanthin and Phytosterols: α- and β-sitosterol, campesterol, α- and β-spinasterol, stigmasterol, cycloartenol and esters thereof, coumarins such as medicagol, coumestrol, savitol, trifoliol, lucernol, triterpene saponins such as medicagosides, medicagenic acid, soyasapogenols, hederagenin, flavonoids, isoflavonoids, isoflavons such as tricin, biocanin, diadzein, formononetin, genistein, coumesterol, sativan, 5′methoxy-sativan, antibiotics, anti-inflammatory compounds and phytoestrogens.

The chlorophyll concentrate may also be used as an animal feed additive (e.g. fodder pellets or silage) or in a fermentation process (please see below).

The at least one high value product obtainable by the method of the present invention may be used as pigments (natural food colours), for the reduction of the cholesterol level, as a vitamin K antagonists, as antioxidants, as phytoestrogens. They may also be used in cosmetic products as hair care, anti-aging products, fragrances and tonics, in dietary supplement for e.g. cholesterol- and blood sugar control, as antibiotics, as anti-inflammatory compounds and as immune system enhancing phytoestrogens.

Pre-Treatment and Utilisation of the Remaining Liquid.

Following the removal of the protein from the clear juice, the remaining liquid—i.e. the brown juice may be concentrated by removing water (e.g. by evaporation). The brown juice may be used in a biogas plant for production of methane.

The concentrated brown juice preferably has a carbohydrate content in the range of 2 to 5% (w/w), such as in the range from 3 to 4% (w/w) thus, it may be used in a subsequent fermentation process. Such fermentation process may an alcohol, amino acid, organic acid, enzyme or methane fermentation.

The alcohol fermentation may be performed by one or more microorganisms selected from the group consisting of yeast and bacteria.

The alcohol fermentation provides a fermentation broth comprising at least 2% (w/w) alcohol and an alcohol fermentation effluent. The fermentation broth may comprise at least 2% (w/w) ethanol, such as at least 5% (w/w) ethanol.

The commercially valuable alcohol may be isolated from the fermentation broth by a distillation.

The alcohol fermentation effluent may be subjected to a methane fermentation process.

Proteins and other nitrogen containing compounds are in anaerobic fermentation processes, converted to ammonia. Ammonia provides an inhibitory effect on the anaerobic fermentation process thus inhibiting the methane production. By using brown juice, where some og the proteins are removed or the alcohol fermentation effluent of the present invention (i.e. comprising a low protein and nitrogen content) in the methane fermentation process, the inhibition caused by ammonia will be either partly or fully reduced.

The anaerobic fermentation process may result in a combustible fuel or gas, such as methane, and an anaerobic fermentation effluent. This anaerobic fermentation effluent may comprise a high content of potassium and can be used as fertilizer.

Additionally the press cake (I) and/or the chlorophyll concentrate (II) or residues from the press cake and chlorophyll concentrate after isolation of high value product may added to the methane fermentation process and/or used in the alcohol fermentation process (i.e. as a 2^{nd} generation biorefinery). In order to provide more efficient processes these solid phased may be subjected to a pre-treatment resulting in a partially separated material. Such pre-treatment may comprises a wet oxidation or a steam explosion. The partially separated material is subjected to a hydrolysis selected from the group consisting of an enzyme hydrolysis, an acid hydrolysis or an alkaline hydrolysis resulting in a slurry containing fermentable sugars. Such slurry may be added to the alcohol fermentation process and/or to the methane fermentation process. The effluent from the ethanol fermentation may also be used in the anaerobic methane fermentation.

A fertilizer comprising a high content of potassium may be obtained from the effluent from the anaerobic digestion mentioned above. The fertilizer may be used as it is or in combination with compost and other organic based fertilizers.

General

In the present context, the expression “liquid phase” is used interchangeable with the expression “juice” and relates to the phase or fraction of the disintegrated plant material after the solid plant material has been removed or partially removed.

The expression “solid phase” or press cake relates in the present context to the phase of the processed plant material after the original liquid or juice has been removed or partially removed, e.g. by a process described above.

In the present context, the expression “native protein” relates to a protein in its natural state, in the cell, unaltered by heat, chemicals, enzyme action, or the exigencies of extraction.

In an embodiment the invention pertains to a leaf protein feed concentrate obtainable by the method according to the present invention. In another embodiment the invention pertains to the use of such leaf protein feed as feed additive in feed mixtures. In a further embodiment the invention pertains to a Soluble functional protein obtainable by the method according to the present invention. In an embodiment the invention pertains to the use of such soluble functional protein as a feed ingredient, as a pharmaceutical ingredient, a cosmetic ingredient and/or as a vegetable protein. In a further embodiment the invention pertains to a green leaf protein concentrate and/or a brown juice obtainable by the method according the present invention and to the use of such green leaf protein concentrate and/or brown juice acas an animal feed or for further isolation of high value products. In further embodiments the invention pertains to a fermentation product and/or a fermentation effluent obtainable by the method of the present invention and to the use of such a fermentation effluent in biogas fermentation or as a fertilizer.

Reference to prior art in this specification is not, and should not be taken as, an acknowledgment or any form of suggestion that this prior art forms part of the common general knowledge in any country.

The reference cited in the present application, are hereby incorporated by reference in its entirety.

As will be apparent, preferred features and characteristics of one aspect of the invention may be applicable to other aspects of the invention. The invention may be embodied in other specific forms without departing from the spirit or essential characteristics thereof. The foregoing embodiments are therefore to be considered in all respects illustrative rather than limiting on the invention described herein. Scope of the invention is thus indicated be the appended claims rather than by the foregoing description, and all changes that come within the meaning and range of equivalency of the claims are intended to be embraced by reference therein.

It should be noted that embodiments and features described in the context of one of the aspects of the present invention also apply to the other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. In addition, the terms “at least one” and “one or more” is in this specification used interchangeably.

The invention will hereinafter be described by way of the following non-limiting figures and examples.

### FIGURE LEGENDS

FIG. 1 represents a schematic overview of a method producing at least one functional protein, other high value products, feed products and fermentation products.

Referring to FIG. 1, the plant material, preferably freshly harvested green biomass (1) is subjected to pressing, in preferably a screw press (2) obtaining a press cake (3) used as animal feed, fibres or bioenergy (5) and a liquid phase, the green juice (4). The green juice is subjected to UHT sterilization/pasteurization (6) and separation (e.g. centrifugation) (7) and separated into solid phase, a green chlorophyll concentrate (9) used as feed additive (10) and isolation of phytochemicals (11) plus a clear juice (8). A soluble functional protein product (14) is separated from the liquid by lowering pH by addition of acid or lactic acid fermentation and separation (e.g. centrifugation), by chromatography or by ultrafiltration or a combination thereof (12). Such precipitation includes acid precipitation. In preferred embodiments the acid precipitation agent is selected from a group consisting of H2SO4, H3PO4 CH3CHOHCOOH, CH3COOH, HCOOH, or combinations thereof or lactic acid fermentation. The recovered functional protein product (14) may be isolated by separation (e.g. centrifugation), filtration or a combination thereof creating a supernatant comprising a low protein content, the brown juice (13). The brown juice are used for isolation of soluble phytochemicals (11) and fermention (15), producing biogas (Methan) or Amino acids, organic acids etc. (17)

Alternatively a feed grade green, leaf protein concentrate is recovered directly from the green juice by lowering pH with acid addition or lactic acid fermentation followed by separation (e.g. centrifugation). For organic farming lactic acid fermentation is used, as addition of inorganic acid is not permitted and because the content of living pro-biotic lactic bacteria in the feed is preferred.

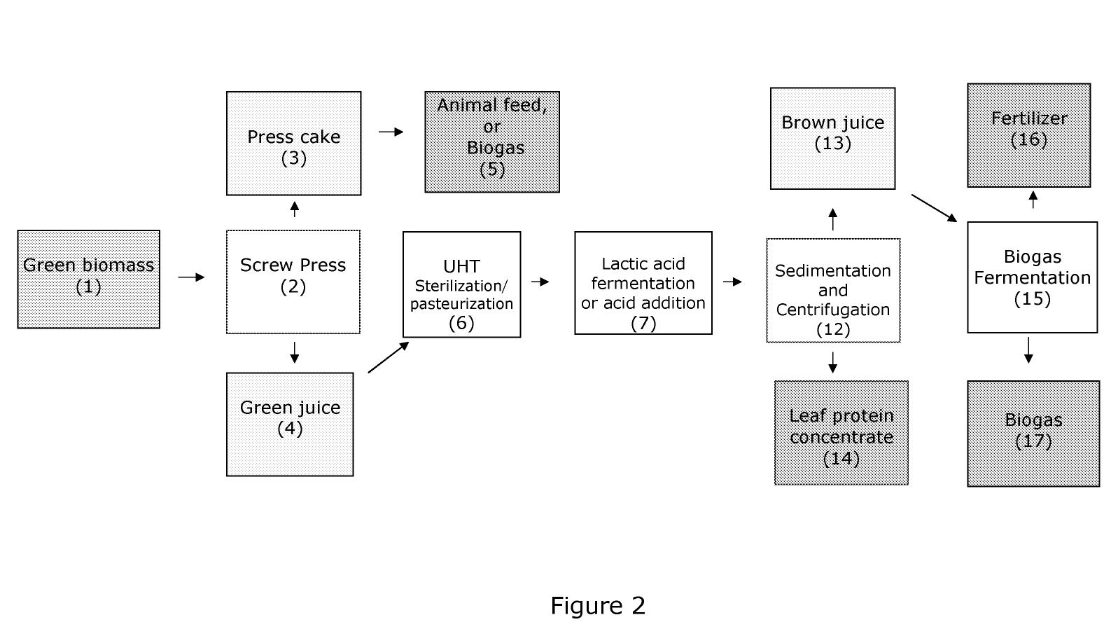

FIG. 2 represents a schematic overview of a method producing a leaf protein feed concentrate.

Referring to FIG. 2, the organically or traditionally grown green biomass is (1), is subjected to pressing, in preferably a screw press (2) obtaining a press cake (3) used as animal feed, fibres or bioenergy (5). The green juice (4) is used as is, fresh or subjected to UHT sterilization/pasteurization (6) followed by (batch or continuous) lactic acid fermentation or addition of acid to pH below 4.0 (7). The fermented or acidified green juice is subjected to sedimentation and separation (e.g. centrifugation) (12) and separated in a leaf protein concentrate (14) and brown juice (13).

The lactic acid fermented brown juice is used in biogas fermentation (15) with production of biogas (17) and Fertilizer (16)

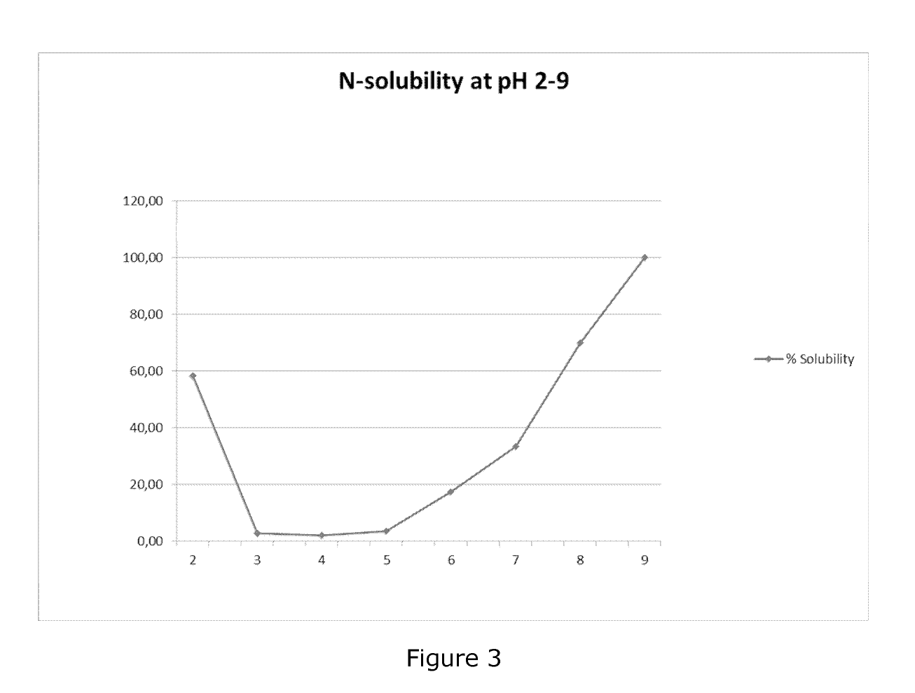

FIG. 3. Referring to FIG. 3 in example 3, the nitrogen solubility profile of the soluble functional protein is measured between pH 2 and 9. 400 mg protein is mixed with 30, 0 ml distilled water in a 50 ml centrifuge tube. The mixture is mixed on a magnetic stirrer in 30 minutes while pH is adjusted with either 1 M NaOH or 1 M HCl to the desired pH. The volume is hereafter adjusted to 40 ml and the sample is centrifuged at 10000×g in 10 minutes. Total N (Kjeldahl) is measured on 10 ml of the supernatant. 100% solubility corresponds to 1% (w/v). FIG. 3 show the nitrogen solubility of acid precipitated alfalfa soluble functional protein.

The invention will now be described in further details in the following non-limiting examples

### EXAMPLES

### Example 1

Isolation of a soluble functional protein from alfalfa juice by freezing, thawing and centrifugation followed by precipitation.

Alfalfa is harvested with a scythe in the early blooming period (June 2009). The freshly harvested alfalfa is disintegrated in a knife mill and added to a screw press. The disintegrated biomass is separated in green juice and a solid phase (I), the pulp.

The green juice is added to 2, 5 litre PE bottles and immediately cooled down to −20° C. by placing the bottles in a bath with dry ice and kept in a freezer at −20° C. After thawing the green juice is kept cold on ice bath (approximately 2-5°) and centrifuged in a cooling centrifuge Beckman Coulter Allegra 25 R at 13.000×g for 10 minutes.

The green juice is separated into a solid phase (combination of phase II and III) and a supernatant, the clear juice.

The clear juice is kept cold on ice bath juice and immediately, under stirring, added sulphuric acid, 10% to pH 4, 0. After 30 minutes at 2-5° C. the functional protein product is separated in a cooling centrifuge, Beckman Coulter Allegra 25 R at 1500×g and washed once in the centrifuge with cold (approximately 2-5° C.) water, acidified to pH=4.0 with sulphuric acid and finally dried in a Heto freeze dryer Maxi Dry Plus.

The solid phase (II+III) (green leaf protein) from the first centrifugation contained 1.355% of the nitrogen (N). The protein content in the combination of solid phase II and III is 35, 5% in dry matter.

27, 4% of the nitrogen (N) in the green juice is precipitated as a functional protein. The protein (N*6, 25) content functional protein product is in this case 76, 9%.

### Example 2

Isolation of a soluble functional protein from leaves of alfalfa by stepwise removal of suspended material, followed by lowering pH to 4.0 and isolation of the functional protein by centrifugation.

Alfalfa is harvested with a scythe in the early blooming period. The freshly harvested alfalfa is disintegrated in a knife mill and added to a screw press. The disintegrated biomass is separated in green juice and a solid phase (I), the pulp. The green juice is added to 2, 5 litre PE bottles immediately cooled down to −20° C. putting the bottles in a bath with dry ice and kept in a freezer at −20° C. After thawing the green juice is kept cold on ice bath (approximately 2-5°). 1, 0 litre (1.022 kg) of the green juice is centrifuged in a cooling centrifuge at 3.500 g for 10 minutes. A solid phase (II) comprising green protein and fibre of 327 g (DM=8, 27%) is separated from the supernatant.

The green supernatant from the centrifuge is thereafter micro-filtered in laboratory scale, using a cross flow filter unit with a 0, 45 μm filter. The permeate is a clear juice, 118 g, (DM=3, 58%) containing the soluble proteins. The retentate i.e. solid phase (III), 577 g (DM=5, 95%) comprises the chloroplast fraction. The filtration was carried out with cold (0-5° C.) green supernatant and the flux in the cross flow filter started at 15 LMH (L/m2·time) and dropped to about 7 LMH. The clear juice is kept cold on ice bath and added sulphuric acid, 10% to pH 4.0.

The functional protein product is obtained in a cooling centrifuge, Beckman Coulter Allegra 25 R at 1500×g and washed once in the centrifuge with cold (2-5° C.) water, acidified to pH=4.0 with sulphuric acid and dried in a Heto freeze dryer Maxi Dry Plus.

### Example 3

Functional properties of the isolated soluble functional protein from example 1.

The functional properties of the isolated soluble functional protein from example 1 has been tested for water absorption, fat absorption, emulsion activity and emulsion stability, foaming ability and foaming stability at different pH.

TABLE 2

| | | Water | Fat | | |
|---|---|---|---|---|---|
| | Bulk | absorption | absorption | Emulsion | Emulsion |
| | density | ml water/g | ml oil/g | activity | stability |
| Protein | g/ml | protein | protein | % | % |
| Functional | 0.109 | 2.6 | 3.4 | 58.4 | 47.6 |
| protein product | | | | | |
| Soya protein | 0.364 | 5.3 | 1.8 | 64.7 | 63.2 |

Functional properties of the functional protein product from alfalfa compared with commercial soya protein

Bulk Density

The soluble functional protein product is placed in a 25 ml pre weighed graduated cylinder. The cylinder is packed by tapping the cylinder on the bench top 10 times from a height of the sample of 5 cm. The volume of the sample is recorded and the cylinder is weighed. The procedure is repeated twice per sample of protein. The bulk density is expressed as g/ml sample.

Water Absorption

5 ml water is added to 500 mg of the soluble functional protein product in a centrifuge tube. The content is mixed by stirring and sonicated for 1 minute to disperse the sample. The suspension is stirred for 30 minutes at room temperature before centrifugation at 1610×g for 25 minutes. The volume of free water is measured and the water retained in the protein pellet is expressed as ml water absorbed per g protein sample.

Fat Absorption

500 mg of the soluble functional protein product is transferred to a 12 ml centrifuge tube. 3 ml sunflower oil is added and the content is mixed by stirring followed by sonication for 1 minute to disperse the sample. After holding at room temperature for 30 minutes, the tube is centrifugated at 1610×g for 25 minutes. The volume of free oil is measured and the oil retained in the protein pellet is expressed as ml fat absorbed per g protein sample.

Emulsifying Activity (EA) and Emulsion Stability (ES)

Emulsifying activity (EA) and Emulsion stability (ES) are determined in the same procedure. 2, 1 g protein is added 30 ml distilled water and dispersed at 18.000 rpm for 30 seconds, where after 30 ml sunflower oil is added and the blending continued for 1 minute at 22.000 rpm.

The formed emulsion is divided equally in four 15 ml centrifuge tubes. For Emulsifying activity (EA) two of these tubes are centrifuged at 1300×g for 5 minutes. The emulsifying activity is expressed as 100× (the height of the emulsified layer divided with the height of the total content in the tube). Emulsion stability is determined similarly to that of emulsifying activity except that prior to the centrifugation, the emulsion in the centrifuge tubes is heated in a water bath at 80° C. for 30 minutes and cooled to 15° C. The Emulsion stability is measured as 100× (the height of the emulsified layer after heating divided with the height of the total content in the tube).

TABLE 3

| | pH 4.0 | | pH 7.0 | | pH 10.0 | |
|---|---|---|---|---|---|---|
| | % volume | % foam | % volume | % foam | % volume | % foam |
| | in- | stabil- | in- | stabil- | in- | stabil- |
| | crease | ity | crease | ity | crease | ity |
| Funct- | 72 | 61.5 | 87 | 40.9 | 93 | 0 |
| ional | | | | | | |
| protein | | | | | | |
| product | | | | | | |
| Soya | 0 | 0 | 50 | 12.5 | 25 | 10 |
| protein | | | | | | |

Foaming capacity and foaming stability (1 hour) of the functional protein product and commercial soya protein at different pH values

Foaming Capacity and Foaming Stability

Whip ability and foam stability are determined in an aqueous 2% protein solution at pH values 4, 7 and 10. 1, 2 g protein is added water and the desired pH is reached by adding 0, 1 M NaOH or 0, 1 M HCl. Working volume is 60 ml. The solution at the desired pH is whipped at 18.000 rpm for 2 minutes in a Warring Blender. Immediately after blending the solution is transferred to a graduated cylinder and the total volume as well as the foam volume is noted. The volume of foam is measured after 15, 30, 60, 80 and 120 minutes. Foaming capacity is calculated as: % volume increase=((volume after whipping−60 ml)/60 ml)×100. Foaming stability is foam volume remaining after a holding time or as % of initial foam volume.

Nitrogen Solubility

The nitrogen solubility profile is measured between pH 2 and 9. 400 mg protein is mixed with 30, 0 ml distilled water in a 50 ml centrifuge tube. The mixture is mixed on a magnetic stirrer in 30 minutes while pH is adjusted with either 1 M NaOH or 1 M HCl to the desired pH. The volume is hereafter adjusted to 40 ml and the sample is centrifuged at 10000×g in 10 minutes. Total N (Kjeldahl) is measured on 10 ml of the supernatant. 100% solubility corresponds to 1% (w/v). FIG. 3 show the nitrogen solubility of acid precipitated alfalfa white protein.

### Example 4

Pilot Plant Trials on Isolation of a Soluble Functional Protein from Alfalfa

Fresh alfalfa has a dry matter content of 31%. The alfalfa is harvested in North Holland (October 25th 2011, temperature outside 10-13° C.) by a mowing machine, and then cut in a knife cutter to 3-5 cm length and transported to the pilot plant, where it is immediately washed in cold water (water added ice cubes to a temperature of about 5° C.). After washing the dry matter content is 16, 0% w/w. During washing the temperature is kept below 9° C. After washing and removal of dirt and sand the alfalfa is disintegrated in a refiner, followed by passing through a screw press.

3333 kg washed, wet fresh alfalfa is separated in this way in 2849 kg green juice with a dry matter of 11.6% w/w and 482 kg press cake (solid phase (I)) with a dry matter content of 42% w/w. The green juice is treated in two ways.

1. The green juice is cooled in an ice bath (0-5° C.) and sent through a decanter operating at 3720×g. The solid phase (II) from the decanter comprises cell debris and fibres. The green supernatant from the decanter has been further micro-filtered in laboratory scale, using a cross flow filter unit with a 0, 45 μm filter. The permeate is a clear juice, containing the soluble proteins. The retentate (solid phase (III)) comprises the chloroplast fraction. The microfiltration was carried out with cold (0-5° C.) juice and the flux in the cross flow filter starting at 15 LMH (L/m2·time).

2. In a second trial the green juice is added sulphuric acid to pH 3, 85 and after storage in a tank for 30 minutes passed through a decanter at 3720×g. The supernatant is a clear brown juice containing the compounds soluble at pH 3, 85, amino acids, organic acids, carbohydrates like fructose, glucose and sucrose and inorganic salts. The solide phase (feed protein (10)) comprises proteins mixed with fibres and other high molecular weight compounds. The dry matter in the feed protein after the decanter is 26, 3% w/w. The protein content in the feed proteinis 45.2% in DM. The feed protein product can be used as it is or dried for animal feed for pigs, cows and chicken.

It can be seen from the pilot scale trial that the potential yield of the functional protein product is about 6.6% dry matter on alfalfa dry matter, corresponding to 660 kg in 10 tons alfalfa dry matter.

### Example 5

Separation of Alfalfa Plants in Stem and Leaves.

Alfalfa is harvested with a scythe before blooming (June 15th 2010), height of plants is between 25 and 30 cm. The leaves are stripped from the stems by hand, reaching an amount of 280 gram of leaves and 229 gram of stems in total 55% leaves.

The leaves is washed and dried in a salad sling, thereafter passed two times in a meat grinder.

The grinded leaves are separated in green juice and a solid phase (I) comprising e.g. fibres by manually pressing alfalfa mash through a stainless steel filter with a mask size of 1, 0 mm. The result is 44 g of green juice with a dry matter content of 9, 9% w/w, 141 gram of fibres with a dry matter content of 17, 4% w/w and a loss of 6 gram. The protein content in the green juice is 22, 7% w/w.

### Example 6

Isolation of Leaf Protein Feed Concentrate from Red Clover

Red clover was harvested on the 14th of May 2014; at Vamdrup, Denmark. Right after the harvest, the biomass was screw pressed obtaining a solid and a liquid fraction, namely press cake and green juice; respectively. The pressing was done using a Vincent CP4 screw press. 62.2 kg freshly harvested red clover were pressed, obtaining 37.3 kg green juice and 24.9 kg press cake. 3.5 kg biomass was lost during the mechanical process. The temperature and pH of the green juice after the mechanical separation were around 14.8° C. and 6.2, respectively. After the solid-liquid separation, the green juice was inoculated with an overnight culture of Lactobaciluus salivarius BS 1001 (20 ml per liter of green juice) and fermented at 38° C. overnight. At that time, the pH was 4.7 and the proteins were precipitated obtaining two fractions, namely brown juice and a leaf protein concentrate. In order to separate those two fractions, a centrifugation step (Centrifuge Beckman GS-6 cooling centrifuge equipped with a GH-3.8 horizontal rotor) was performed during 10 minutes at 3800 rpm and 5° C.

Chemical Composition of the Different Fractions and Mass Balances

The chemical composition of the different fractions obtained during the biorefinery process is presented in Table 1. The dry matter content of the fresh material was 164 g/kg, where organic matter represented 88%. Dry matter content in the press cake after the mechanical fractionation was increased 1.5-fold, when compared with the fresh biomass. The proteins in the press cake represented around 18% (dry matter basis), which was similar to the approx. 20% protein content in the fresh material on a dry matter basis. Most of the free sugars in the fresh biomass were recovered in the green juice after the mechanical fractionation. Those free sugars were fermented into lactic acid, decreasing the pH to final values of 4.7 and 4.3, in the brown juice and protein concentrate, respectively. The protein concentrate presented a dry matter of 191 g/kg, of which 93% was organic. The crude protein content of the protein concentrate was 39.3% (dry matter basis).

TABLE 1
Chemical composition of the different fractions obtained during the biorefining process.

| | | Fresh material | | Press cake | | Green juice | | Brown juice | | Protein concentrate | |
|---|---|---|---|---|---|---|---|---|---|---|---|
| Parameters | | Average | STD | Average | STD | Average | STD | Average | STD | Average | STD |
| pH | | 5.7 | n.d. | 5.5 | n.d. | 5.0 | n.d. | 4.7 | n.d. | 4.3 | n.d. |
| Total solids | g/kg | 163.6 | 9.6 | 236.6 | 3.4 | 62.2 | 0.8 | 24.6 | 0.1 | 191.3 | 1.4 |
| Volatile solids | g/kg | 144.5 | 9.2 | 217.7 | 2.8 | 51.0 | 0.9 | 18.8 | 0.1 | 178.2 | 1.3 |
| Ash | g/kg | 19.1 | n.d. | 18.9 | n.d. | 11.2 | n.d. | 5.8 | n.d. | 13.1 | n.d. |
| Total Kjeldahl | g/kg | 5.3 | 0.2 | 7.0 | 0.2 | 2.5 | 0.1 | 0.8 | 0.1 | 12.0 | 1.5 |
| Nitrogen | | | | | | | | | | | |
| Crude protein | g/kg | 33.1 | n.d. | 43.6 | n.d. | 15.7 | n.d. | 4.8 | n.d. | 75.1 | n.d. |
| Free sugars | g/kg | 23.0 | 2.7 | n.d. | n.d. | 23.6 | 0.03 | 7.8 | 0.22 | 15.3 | 0.2 |
| Lactic acid | g/kg | 0.0 | 0.0 | 0.0 | 0.00 | 0.0 | 0.00 | 6.8 | 0.24 | 14.8 | 0.1 |

n.d. stands for not determined

Mass balances are presented in Table 2. Although some loss of material was observed during the process, it was decided not to take them into account since they will be minimized when scaling up the biorefinery process. According to the mass balance based on fresh biomass, 60% of the fresh weight mass was recovered as green juice, obtaining 67 kg of leaf protein feed concentrate per tonne of fresh red clover biomass. 72% and 14% of the dry matter was recovered in the press cake and brown juice, respectively, which is convenient, as these two fractions will be used to produce biogas. 65% of the crude protein in the fresh biomass was recovered as fiber-bound protein in the press cake, whereas 23% of the crude protein in the fresh biomass was recovered in the leaf protein feed concentrate.

TABLE 2
Mass balances for red clover separation-protein precipitation.

| | Fresh biomass | Dry matter | Crude protein |
|---|---|---|---|
| | kg/t | kg/t | kg/t |
| Fresh material | 1000 | 1000 | 1000 |
| Press cake | 401 | 718 | 649 |
| Green juice | 599 | 282 | 351 |
| Brown juice | 532 | 142 | 117 |
| Protein concentrate | 67 | 140 | 234 |

Amino Acid Profiles

The amino acid profile in the fresh material, green juice and protein concentrate was analyzed and it is presented in Tables 3 and 4. The data corresponding to the amino acids tryptophan and tyrosine are not included since the method that was used does not measure these two amino acids. The amino acid profile in terms of grams of amino acid per kilogram of true protein (i.e. sum of each amino acid concentration) was in the same range for the fresh material and the protein concentrate. However, it was slightly lower in the green juice, with exception of asparagine which up-concentrated compared with the fresh crop and the protein concentrate (Table 3). This fact explains that the amino acids contained in the fresh material were recovered during the biorefinery process ending up in the protein concentrate. The three fractions presented a balanced content of amino acids.

Table 4 presents the amino acid concentration on dry matter basis. As expected, the amino acid concentration in the leaf protein feed concentrate increased up to 2.7 times when compared to the fresh material. The amino acid profile in the protein concentrate is comparable with an organic basal diet for poultry and with the soybean meal, which is the main source of protein for organic monogastric livestock. The concentration of essential amino acids such as methionine is higher than in the commercial organic basal diet and in the same range as the soybean meal.

TABLE 3
Amino acid profile in the different fractions through the biorefinery process.

| | Arg | His | Ile | Leu | Lys | Met | Phe | Thr |
|---|---|---|---|---|---|---|---|---|
| | g/kg TP | g/kg TP | g/kg TP | g/kg TP | g/kg TP | g/kg TP | g/kg TP | g/kg TP |
| Fresh material | 58.8 | 23.8 | 52.3 | 89.8 | 65.7 | 17.7 | 58.5 | 50.2 |
| Green juice | 42.1 | 18.9 | 47.5 | 78.2 | 57.1 | 14.7 | 50.3 | 46.5 |
| Protein concentrate | 67.7 | 27.3 | 59.2 | 99.4 | 67.7 | 21.3 | 66.9 | 50.8 |
| | Val | Ala | Asp | Cys | Glu | Gly | Pro | Ser |
| | g/kg TP | g/kg TP | g/kg TP | g/kg TP | g/kg TP | g/kg TP | g/kg TP | g/kg TP |
| Fresh material | 65.9 | 63.5 | 171.7 | 8.0 | 115.8 | 55.8 | 49.6 | 53.1 |
| Green juice | 61.2 | 64.8 | 255.7 | 6.6 | 110.1 | 51.4 | 45.5 | 49.4 |
| Protein concentrate | 71.2 | 66.2 | 121.7 | 6.4 | 118.9 | 59.0 | 48.3 | 48.1 |

TP stands for true protein

TABLE 4
Amino acid composition of fresh crop, green juice and protein concentrate of red clover. Amino acid composition
for organic basal diet for poultry (Hammershøj and Steenfeldt, 2012) and soybean meal (Sriperm et al., 2011).

| | Arg | His | Ile | Leu | Lys | Met | Phe | Thr |
|---|---|---|---|---|---|---|---|---|
| | g/kg DM | g/kg DM | g/kg DM | g/kg DM | g/kg DM | g/kg DM | g/kg DM | g/kg DM |
| Fresh material | 10.2 | 4.1 | 9.1 | 15.6 | 11.4 | 3.1 | 10.2 | 8.7 |
| Green juice | 7.5 | 3.4 | 8.5 | 14.0 | 10.2 | 2.6 | 9.0 | 8.3 |
| Protein concentrate | 26.8 | 10.8 | 23.5 | 39.4 | 26.8 | 8.5 | 26.5 | 20.1 |
| Organic basal diet | 11.5 | 4.9 | 8.7 | 16.7 | 9.3 | 3.6 | 9.8 | 7.5 |
| Soy bean | 37.4 | 13.5 | 23.1 | 39.0 | 32.3 | 7.7 | 26.5 | 20.2 |
| | Val | Ala | Asp | Cys | Glu | Gly | Pro | Ser |
| | g/kg DM | g/kg DM | g/kg DM | g/kg DM | g/kg DM | g/kg DM | g/kg DM | g/kg DM |
| Fresh material | 11.5 | 11.0 | 29.8 | 1.4 | 20.1 | 9.7 | 8.6 | 9.2 |
| Green juice | 11.0 | 11.6 | 45.8 | 1.2 | 19.7 | 9.2 | 8.2 | 8.9 |
| Protein concentrate | 28.2 | 26.3 | 48.2 | 2.5 | 47.1 | 23.4 | 19.2 | 19.1 |
| Organic basal diet | 9.4 | 9.8 | 17.0 | 3.6 | 41.3 | 8.9 | 13.9 | 10.3 |
| Soy bean | 24.1 | 22.4 | 59.5 | 6.9 | 92.5 | 21.6 | 24.1 | 25.8 |

Final Remarks and Future Perspectives

Up to 67 kg of protein concentrate per tonne of fresh red clover is obtained after the biorefining process. With a dry matter content around 200 g/kg this would lead to 13-14 kg of dry protein concentrate. This dry organic product contains 40% of crude protein and a balanced amino acid profile, comparable with the soybean meal.

Moreover, two different sub-products are obtained during the biorefinery process, namely press cake and brown juice. The high content in lignocellulosic material and fiber-bounded protein in the press cake makes it very likely a suitable product to be used as feed for ruminants. Both sub-products are also adequate to be used as substrate for biogas production since the brown juice presents high content of minerals and easily degradable organic matter while the press cake presents high organic matter content and proteins. Finally, the organic digestate obtained after the anaerobic digestion of the sub-products could be utilized as organic fertilizer.

### REFERENCES

Gibbson, W. R., Westby, C. A. and Arnold, E. 1987. Semicontinuous diffusion fermentation of fodder beets for fuel ethanol and cubed protein feed production. Biotechnology and Bioengineering, 31, pp. 696-704.

1-15. (canceled)

16. A method for providing a chlorophyll concentrate and/or at least one soluble functional protein said method comprises the steps of:
(i) pressing a green plant material;
(ii) obtaining a press cake and a green juice;
(iii) subjecting said green juice to UHT Sterilization/pasteurization and obtaining a sterilized green juice;
(iv) subjecting the sterilized/pasteurized green juice to separation and obtaining a chlorophyll concentrate and a clear juice; and
(v) recovering at least one soluble functional protein from the clear juice.

17. A method for providing a leaf protein feed concentrate said method comprises the steps of:
(i) pressing a green plant material;
(ii) obtaining a press cake and a green juice;
(iii) optionally subjecting said green juice to UHT Sterilization/pasteurization and obtaining a sterilized/pasteurized green juice;
(iv) subject the green juice or the sterilized green juice to lactic acid fermentation followed by separation; and
(v) obtaining a leaf protein feed concentrate and a brown juice.

18. The method according to claim 16, wherein said plant material comprises a protein content in the range from 0.1 to 6% (w/w).

19. The method according to claim 17, wherein said plant material comprises a protein content in the range from 0.1 to 6% (w/w).

20. The method according to claim 16, wherein said plant material is selected from the group consisting of alfalfa, clover, grass, beet, chicory, Jerusalem artichoke, sugar cane, carrot, radish, roadside crops and combinations hereof.

21. The method according to claim 17, wherein said plant material is selected from the group consisting of alfalfa, clover, grass, beet, chicory, Jerusalem artichoke, sugar cane, carrot, radish, roadside crops and combinations hereof.

22. The method according to claim 17, wherein the method further comprises a step of fermentation of the brown juice and obtaining a fermentation product and a fermentation effluent.

23. The method according to claim 16, wherein the green juice has protein content in the range of 0.1 to 15% (w/w).

24. The method according to claim 17, wherein the green juice has protein content in the range of 0.1 to 15% (w/w).

25. The method according to claim 16, wherein the centrifugation is performed at 2-10,000×g.

26. The method according to claim 17, wherein the centrifugation is performed at 2-10,000×g.

27. The method according to claim 16, wherein the green supernatant has protein content in the range of 0.1 to 15% (w/w).

28. The method according to claim 17, wherein the green supernatant has protein content in the range of 0.1 to 15% (w/w)

29. The method according to claim 16, wherein the clear juice has a protein content in the range from 0.5 to 10% (w/w).

30. The method according to claim 17, wherein the clear juice has a protein content in the range from 0.5 to 10% (w/w).

31. The method according to claim 17, further comprising a step wherein the leaf protein feed concentrate is dried and obtaining a dry protein feed comprising living pro-biotic lactic acid bacteria.
